(19) United States
(12) Patent Application Publication
(10) Pub. No.: US 2004/0157774 A1
(43) Pub. Date: Aug. 12, 2004

(54) Epitope-tagged recombinant Growth Arrest Specific Gene 6 protein

(76) Inventors: Jing Yang, Ambler, PA (US); Cailin Chen, New Hope, PA (US); Bing Hu, Media, PA (US); Patricia Andrade-Gordon, Doylestown, PA (US); Robert Jordan, Thornton, PA (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/671,054

(22) Filed: Sep. 24, 2003

Related U.S. Application Data
(60) Provisional application No. 60/413,157, filed on Sep. 24, 2002.

(51) Int. Cl. A61K 38/17; C07K 14/705

(52) U.S. Cl. 514/12; 530/350

(57) ABSTRACT

The present invention provides epitope-tagged mammalian recombinant Growth Arrest Specific Gene 6 (Gas6) proteins or polypeptides that retain their biological activity of Gas6, including a novel method of generating and purifying the aforesaid proteins or polypeptides. Such biolgically active eptitope-tagged Gas6 proteins or polypeptides can be used when the presence of Gas6 is required and/or when Gas6 antagonists and receptors need to be identified. Prior to being expressed from a mammalian cell, the recombinant Gas6 protein or polypeptide is tagged with either Flag or polyhistidine to the C-terminus. After expression, the protein or polypeptide is purified in a single step affinity purification using either anti-Flag antibody resin or nickle resin. The purified epitope-tagged mammalian recombinant Gas6 protein or polypeptide maintains its biological activity and can be used thereafter.

### CROSS REFERENCE TO RELATED APPLICATION

This application claims priority of U.S. provisional application serial No. 60/413,157, filed Sep. 24, 2002.

### BACKGROUND OF THE INVENTION

A. Field of the Invention

The present invention relates to epitope-tagged mammalian recombinant Growth Arrest Specific Gene 6 (Gas6) protein or polypeptide that retains the biological activity of Gas6, including a method for generating and purifying at least one such epitope-tagged Gas6 protein or polypeptide, as well as using the epitope-tagged recombinant Gas6 protein or polypeptide where the presence of Gas6 is required and/or in assays for identifying antagonists for Gas6 or its receptors.

B. Related Art

Gas6 was first identified among a set of genes that are highly expressed during serum starvation of NIH 3T3 cells [1]. It was later classified as a new member of a vitamin K-dependent protein family closely related to the coagulation factor Protein S [2] [U.S. Pat. No. 5,538,861]. Gas6 and Protein S share common structure features with ˜40% sequence homology. Both proteins contain a γ-carboxyglutamic acid-rich domain (Gla domain) at the N-terminus followed by four Epidermal Growth Factor-like (EGF) repeats and a tandem globular domain (G domain). However, Gas6 lacks the thrombin recognition sites that are important for the coagulation activity of Protein S. The G domain of Gas6 belongs to a superfamily of proteins including basement membrane proteins laminin and agrin and human steroid hormone-binding globulin (or androgen-binding protein) [3].

Gas6 is a putative ligand for the Axl family of receptor tyrosine kinases including Axl (Ark, Ufo, Tro7) [4, 5, and U.S. Pat. No. 5,538,861], Mer (Eyk, Nyk) [6] and Sky (Rse, Tyro3, DtK, Brt, Tif) [7, 8]. Gas6 binds to the receptors with nanomolar affinity and causes receptor auto-phosphorylation. The ability of Gas6 to bind to and activate the receptors requires vitamin K-dependent γ-carboxylation [9, 10]. However, truncated Gas6 or a splice variant of Gas6 containing the G domain is sufficient to activate the Rse receptor [11, 12] [U.S. Pat. No. 6,211,142]. It seems that the G domain is masked without γ-carboxylation. This “mask” can be removed by properly modified Gla domain or removal of sequence upstream of the G domain. Gas6 is often found to be associated with membrane due to Gla-mediated calcium-dependent binding to membrane phospholipids [13]. In most systems, secreted Gas6 or soluble recombinant Gas6 has full biological activity, although it is unknown if Gas6 is anchored to the membrane during receptor activation. One exception is that 3T3 cells transfected with Gas6, but not soluble Gas6, support growth of hematopoietic progenitor cells [14]. The hematopoietic effect of the transfected cells is not dependent on vitamin K and the nature of membrane association of Gas6 and the mechanism of action is unexplained.

Gas6 is expressed in the lung, intestine and terminally differentiated cells of most organs including capillary endothelial cells, vascular smooth muscle cells (VSMC) and neurons [2, 15]. It is also found in the alpha granules of platelets that are secreted or transported to the cell surface upon activation [16, 17]. Gas6 is not detected in plasma, macrophages, basophils, neutrophils, or lymphocytes. Under pathological conditions, Gas6 is up regulated at sites of inflammation, vessel injury, and in VSMCs of atherosclerotic plaques [18-20]. Axl is expressed by vascular endothelial cells, CD34+progenitor cells, bone 2 0 marrow stromal cells, monocytes and macrophages, but not in granulocytes or lymphocytes [5, 21-23]. Neuronal cells and many peripheral cells also express Axl. Mer is expressed in lung, kidney, ovary, prostate, mononuclear cells, monocytes, and macrophages, but not in granulocytes or peripheral blood B or T cells [24, 25]. While Sky is mainly expressed in adult brain, it is also found in gonadal tissues, kidney, and in regions of lymphoid tissues that exclude B or T cells [26, 27]. All three receptors are found in human platelets [17].

Over the past decade, Gas6 has been implicated in many cellular functions such as cell growth, apoptosis, cell adhesion and migration, phagocytosis, and possibly hematopoiesis. Gas6 is a potent mitogen for human Schwann cells [28]. It has been disclosed that Gas6 can be used to support cultured Schwann cells for the treatment of neuronal injury [U.S. Pat. No. 5,721,139]. Purified Gas6 also induces proliferation of serum-depleted NIH 3T3 cells [29] and density-inhibited C57 mammary cells [30].

Mesangial cell proliferation, a hallmark of glomerular sclerosis, can be stimulated by Gas6 [31]. In a model of glomerulonephritis induced by Thy 1.1 antibody, Gas6 and Axl levels are dramatically increased in mesangial cells. Injection of wafarin, an inhibitor of γ-carboxylation, or an extracellular portion of Axl suppresses mesangial cell proliferation in this disease model, suggesting that Gas6 plays a role in glomerular disease. Proliferation of VSMC and mesangial cells is also a feature of chronic rejection after kidney transplant. Moreover, Gas6 and Sky are highly expressed in normal kidney and the level of Gas6 is significantly higher in kidney tissues of allografts and isografts [32]. Thus, it is possible that Gas6 contributes to the pathogenesis of chronic rejection. In addition, Gas6 can stimulate GnRH neuronal cell migration [33], suggesting a role of Gas6 in the central nerve system.

Under certain conditions, Gas6 prevents cell death. Gas6 rescues human umbilical 10 vein endothelial cells from apoptosis induced by serum-deprivation or tumor necrosis factor α [18]. Gas6 is also a survival factor for several other types of cells under serum deprivation, including NIH 3T3 fibroblast cells [29, 34, 35], cancer cells [36, 37], Gonadoptropin-releasing hormone (GnRH) neuronal cells [38] and hippocampal neuronal cells [39]. In contrast, endothelial cells from Gas6 knockout mice are protected from cell death induced by cytokines and anti-Fas antibodies [WO Patent No. 00/76309]. Also, the lack of Gas6 in mice suppresses angiogenesis induced by VEGF-matrigel. Since the ability of activated endothelial cells to secrete cytokines and growth factors has been greatly compromised in Gas6 knockout mice, Gas6 may contribute to endothelial cell apoptosis and angiogenic by regulating local levels of cytokines and growth factors.

The Axl family of receptors is present in many different types of tumor cells and is implicated in neoplasia. Axl, cloned from patients with chronic myelogenous leukemia (CMA), was the first of the family to be identified [Liu, 1988 #205] [40]. Elevated levels of Axl are associated with metastatic colon cancer and lung cancer [41, 42]. Although none of the receptors or Gas6 are expressed in B and T cells [27], both Axl and Sky are expressed in myeloid leukemic blasts, while Mer is found in neoplastic T- and B-cell lines [21, 24, 43]. Mammary tumors but not non-tumorigenic progenitors express elevated levels of Sky [44]. Axl, Mer, and Sky are all capable of inducing transformation of fibroblasts [40, 45, 46]. Although a high level of Gas6 is found in multiple myeloma [47], the potential neoplastic effects of the receptors may be mediated by other ligand(s) as well. The most convincing evidence for other ligands for these receptors comes from studies of receptor and Gas6 knockout mice. The phenotype in the receptor triple knockout mice is more severe than that in Gas6 knockouts. Mice lacking all three receptors have multiple organ failure, adult blindness, lack of sperm in males and develop severe autoimmunity [27, 48]. Gas6 knockout mice do not show any obvious phenotype unless challenged under pathological conditions such as thrombosis and endothelial activation [17] [WO Patent No. 00/76309]. Given the similarity of Gas6 to Protein S, it is possible that Protein S may have a role in activation of Axl, Mer, and Sky under certain physiological conditions. Protein S appears to bind to Sky in vitro. However, bovine and human Protein S bind well to murine Sky but not to their homologous receptor [4, 7].

Through interaction with Mer, Gas6 seems to play a role in outer segment phagocytosis by retinal pigment epithelial cells. Vertebrate photoreceptors undergo daily phagocytosis of photoreceptor outer segments by the adjacent retinal pigment epithelium (RPE). The Royal College of Surgeons (RCS) rat, with inherited homozygous deletion of Mer, suffers from retinal degeneration due to an inability of RPE cells to clear the outer segment [49]. Phagocytosis of outer segment by cultured rat RPE cells can be stimulated by Gas6 [50]. Mer is also reported to play a role in phagocytosis of apoptotic cells by macrophages, an important process to prevent inflammation and autoimmunity against intracellular antigens [51]. Mutant mice expressing kinase-deleted Mer have increased autoantibodies, and macrophages from the mutant fail to clear apoptotic thymocytes. The phenotype of autoimmune response is more severe in the receptor triple knockout [27]. It is unclear whether Gas6 mediates any of these latter responses through Mer since Gas6 knockout mice are not reported to develop autoimmunity.

Gas6 is expressed in hematopoetic tissues and seems to regulate erythropoiesis under pathological conditions [52, 53]. Gas6 knockout mice have a reduced erythrocyte count and fewer cells of erythroid lineage in bone marrow, spleen, and fetal liver. The hematocrit in the blood is, however, normal in the mutant mice. Gas6 deficient mice are more susceptible to acute hemolytic anemia induced by phenylhydrazine or autoimmune hemolytic anemia induced by NZB-derived 4C8 IgG2a anti-red cell antibody. Even though bone marrow erythroid precursors do not express Gas6, the erythropoietic effect of Gas6 may be mediated through Sky as it is found in erythroid precursors.

Vascular neointima formation, a process involving VSMC proliferation and migration, contributes to the formation and progression of lesions of restenosis and atherosclerosis. Treatment of VSMC with Gas6 causes enhancement of the growth response to thrombin and angiotensin II [54], induction of cell migration [55] and prevention of serum-deprivation-induced cell death [56]. Upon balloon injury, the level of Gas6 and Axl rises dramatically in the rat carotid artery [19]. This up-regulation of Gas6 and Axl parallels the time course of migration of the VSMC from media to intima, suggesting a role of Gas6 in the pathogenesis of restenosis and atherosclerosis. In fact, arterial stenosis induced by carotid artery ligation is reduced in Gas6 deficient mice [WO Patent No. 00/76309].

One of the common features of all cardiovascular disorders is activation of endothelium, which induces inflammatory responses causing severe damage in affected tissues. Gas6 may be involved directly or indirectly in the inflammatory response. A role of Gas6 in leukocyte adhesion during inflammatory response is controversial. In murine myeloid progentitor 32D cells, Gas6 promotes Axl-mediated cell adhesion [57]. A high concentration of Gas6, on the other hand, inhibits granulocyte adhesion to endothelial cells [58]. Interestingly, endothelial cells lacking Gas6 fail to induce expression of cytokines, adhesion molecules and tissue factor upon TNFα or endotoxin stimulation [WO Patent No. 00/76309], indicating a pro-inflammatory role of Gas6. Leukocyte adhesion to the arterial wall upon endotoxin challenge is markedly reduced in Gas6 deficient mice. In an ischemic stroke model, the infarction size in the Gas6 knockout mice is significantly reduced, possibly as a result of suppression of the inflammatory response [WO Patent No. 00/76309].

Studies from Gas6 deficient mice have revealed a surprising function of Gas6 in thrombosis [17]. Platelet aggregation and secretion stimulated by other aganonists are impaired in Gas6 knockout mice. Platelet aggregates induced by thrombin from the mutant are loosely packed, suggesting the possibility that lack of Gas6 might prevent formation of stable platelet plaque in vivo. Indeed, Gas6 contributes to thrombus generation in vivo. Gas6 mutant mice or wild type mice treated with neutralizing polyclonal antibodies are protected from lethal challenge of pulmonary thrombosis, a platelet dependent thrombosis model. Gas6 may also contribute to fibrin dependent thrombus formation due to its effect on tissue factor expression in endothelial cells. This is supported by the result from other thrombosis models in which the role of platelet is less prominent [17]. The thrombus size in Gas6 mutant mice is 60-85% smaller than wild type after carotid artery injury- or ligation of inferior vena cava.

There is a controversial role of Gas6 in diabetes, particularly in the development of noninsulin-dependent diabetes mellitus (NIDDM) and insulin-resistant disorders. Transgenic mice ectopically expressing Axl or the extracellular domain of Axl in myeloid cells develop phenotypes similar to NIDDM [59]. These animals display hyperglycemia, hyperinsulinemia, severe insulin resistance, progressive obesity, hepatic lipidsis and pancreatic islet dysplasia, but do not exhibit hyperphagia. These animals express an elevated level of TNFα in serum, which may cause insulin resistance in these mice. Addition of Gas6 to blood samples eliminates LPS-induced TNFα induction. Transgenic mice systemically expressing Gas6, on the other hand, do not show diabetic phenotype. In a different set of experiments, administration of Gas6 in combination with insulin causes higher insulin level than mice treated with insulin alone [WO Patent No. 99/49894]. Further study is required to support a role of Gas6 in diabetes under physiological condition.

Gas6 and Sky are expressed in osteoclasts and seem to be involved in osteoclastic bone resorption [60]. Treatment with Gas6 doubles the amount of pit area on a denite slice resorbed by osteoclast cells. Coincidentally, the level of Gas6 is up regulated in ovariectomized mice receiving estrogen. Ovariectomized mice is a model of postmenopausal osteoporosis caused by estrogen withdrawal. Osteoclast bone resorption is also observed in rheumatoid arthritis and ostoarthritis, which is accompanied by an elevated level of Gas6 [18]. Thus, it is possible that Gas6 contributes to bone loss in patients suffering from osteoporosis and arthritis.

Overall, Gas6 plays an important role in multiple patho-physiological processes, many of which lead to life threatening diseases. Development of Gas6 antibodies will be useful for a variety of diagnostic applications and a broad spectrum of therapeutic applications. In particular, neutralizing monoclonal antibodies or antagonists of human Gas6 can be applied to prevent or treat thromboembolic disease or thrombotic pathologic condition such as ischemic disease (ischemic stroke, ischemic cerebral infarction, acute myocardial infarction, and chronic ischemic heart disease), venous thromboembolism, arterial or venous thrombosis, pulmonary embolism, restenosis following coronary artery bypass surgery or following percutaneous transluminal angioplasty of a coronary artery, diabetic angiopathy and allograft arteriosclerosis. Gas6 antagonists may also be beneficial for preventing or treating other disease conditions such as cancer, atherosclerosis, sepsis, glomerular sclerosis, diabetes, rheumatoid arthritis, osteoarthritis and osteoporosis.

Gas6 has been previously purified from cells producing endogenous or recombinant Gas6 by a tedious process containing multiple chromotographic steps [5, 8, 54]. It is known in the art to employ an epitope sequence or “tag” that is specifically recognized by a monoclonal antibody of high affinity to purify, characterize or identify a protein composition. An epitope tag construct that does not impair the functionality of the protein is preferable. Recombinant Gas6 with an epitope tag would greatly facilitate protein purification and identification as large quantities of Gas6 selective antibodies are not readily available. An epitope tag gD fused to the N-terminus or C-terminus of Gas6 has been reported previously. Its application, however, is limited to allowing quantitative analysis of expressed Gas6 in the conditioned medium since the tagged protein does not retain biological function [11]. This invention describes a novel epitope-tagged Gas6 polypeptide that maintains Gas6 biological activity and methods for producing such polypeptides. This recombinant polypeptide can be used in compositions where the presence of Gas6 is needed and in assays of identifying antagonists for Gas6 and its receptors.

### SUMMARY OF THE INVENTION

The present invention provides epitope-tagged mammalian recombinant Gas6 proteins or polypeptides that retain the biological activity of Gas6 such as binding to known receptors and inducing receptor activation as well as a novel method for generating the said epitope-tagged recombinant Gas6 protein or polypeptide. This invention thus relates to the generation of epitope-tagged mammalian Gas6 proteins or polypeptides that retain their biological activity and can be used where the presence of Gas6 is required and/or in assays to identify antagonists to Gas6 and its receptors.

In one aspect the present invention provides for attaching a commonly used epitope tag including Flag and/or polyhistidine at the C-terminus of a mammalian Gas6 protein or polypeptide and then expressing the recominant protein or polypeptide in mammalian cells. The mammalian Gas6 DNA may be of any mammal, including but not limited to human, primate, rodent, or murine, or it may be from any cDNA or genomic library. In one embodiment, the Gas6 polypeptide is human Gas6 protein and the epitope tag is a Flag tag attached at the C-terminus of the Gas6 polypeptide.

In another aspect, the present invention provides for obtaining the recombinant epitope-tagged protein or polypeptide by single step affinity purification using an anti-Flag antibody resin or a nickle resin that is inexpensive and readily available.

The present invention further provides, in one aspect, purified epitope-tagged recombinant Gas6 protein or polypeptide that binds to known Gas6 receptors and induces receptor activation.

In another aspect, the present invention provides for using the eptitope-tagged recombinant Gas6 protein or polypeptide where the presense of Gas6 is needed.

In another aspect, the present invention provides for using the epitope-tagged recombinant Gas6 protein or polypeptide in assays to identify antagonists for Gas6 and its receptors.

In another aspect, the invention provides isolated nucleic acid sequences comprising nucleic acid sequences encoding a Gas6 protein operably linked to a nucleotide sequence encoding an epitope tag.

The present invention further provides any invention described herein.

### DESCRIPTION OF THE FIGURES

FIG. 1. Purification of Gas6_Flag. Conditioned media from HEK293 cells expressing human Gas6_Flag were collected and loaded onto an anti-Flag M2 resin. After washing the resin with buffer, Gas6_Flag was eluted by Flag peptide, dialyzed and analyzed by SDS-PAGE. Protein Staining is shown.

FIG. 2. Binding of Gas6_Flag to Axl-Fc receptor. Dose-dependent binding of Gas6_Flag to a dose range of 0.001-2 mg/ml Axl-Fc was determined by an EIA. A 96 well plate was coated with Axl-Fc, blocked with 1% BSA and incubated with Gas6_Flag. The plate was probed with HRP-conjugated anti-Flag M2 antibody followed by treatment with the substrate. The signals at OD₄₉₀ were determined. A. Dose-dependent binding of Gas6_Flag to 0.75 mg/ml Axl-Fc is shown. B. Dose-dependent binding of 50 ng/ml of Gas6_Flag to Axl-Fc is shown. N=3

FIG. 3. Neutralization of Gas6_Flag binding to Axl-Fc receptor by a goat anti-Gas6 antibody. A goat anti-human Gas6 IgG (R&D Systems) was used to inhibit binding of Gas6_Flag to Axl-Fc receptor by an EIA. A 96 well plate was coated with 0.5 μg/ml Axl-Fc (R&D Systems) blocked with 1% BSA and incubated with IgG alone or IgG plus 50 ng/ml Gas6_Flag. HRP-conjugated anti-Flag M2 antibody followed by treatment with the substrate. The signals at OD₄₉₀ were determined. A. Dose-dependent inhibition of Gas6_Flag binding to Axl-Fc is shown. B. Percent inhibition of binding by the goat anti-Gas6 IgG is shown. N=3.

### DESCRIPTION OF THE INVENTION

The present invention provides eptitope-tagged mammalian recombinant Gas6 protein or polypeptide that retains the biological activity of Gas6 as well as a novel method of generating and purifying the aforesaid protein or polypeptide and the uses of such.

A. Generation of Recombinant DNA of Epitope-Tagged Gas6

In the present invention, human Gas6 cDNA was obtained by polymerase chain reaction (PCR) from reverse transcribed human CHRF cDNA. The primers used were: 5′GCTCTAGAAC CATGGCCCCT TCGCTCTCGC3′ and 5′ GCTCTAGAAC AGAGACTGAG AAGCCTGC3′. Gas6_Flag cDNA was then obtained by a second PCR using the same upstream primer and 5′GCTCTAGACT ACTTGTCGTC GTCGTCCTTG TAGTCGGCTG CGGCGGGCTC CACGG as the downstream primer. Finally, to generate the plasmid vector pcDNA3.1/Gas6_Flag, the full-length cDNA was inserted into a pcDNA3.1 plasmid containing a hygromycin resistant gene (Invitrogen, Carlsbad, Calif.).

In the present invention, rats or other mammals may also be used to generate the epitope-tagged recombinant Gas6 cDNA. This may be accomplished for examply when rat Gas6 cDNA is PCR purified from a rat heart Quick-cDNA library (Clontech, Palo Alto, Calif.) using such primers as 5′XbaI-GGCC TCGCCATGCC GCCAC3′ and 5′XbaI-GGCT GTGACGTGCT CCACAGG3′. The PCR product may be used to create a palsmid vector containing the Flag epitope tag as described above.It is noted that it is important for the resulting product to retain biological activity. This was not the case when the rat Gas6 cDNA product was cloned at the XbaI site of pcDNA3.1_Myc-His vector resulting in in-frame fusion of Gas6 to the Myc-His tag at the C-terminus. The resulting plasmid vector was pcDNA3.1/Gas6_Myc-His.

As an alternative to either method, the DNA encoding Gas6 polypeptides may be obtained by DNA synthesis, from any cDNA library prepared from tissues believed to possess the Gas6 mRNA, or from a genomic library. The nucleic acid sequences can be cloned by PCR amplification or by the traditional method of DNA hybridization and expression cloning.

A human Gas6 polypeptide employed in the present invention can include variants containing one or more amino acid substitutions, deletions or additions, either from natural mutations or human manipulation, as specified herein. Such mutations or substitutions result in muteins, whose mutations can be significant enough to alter the properties of the peptide.

Of course, the number of amino acid substitutions a skilled artisan would make depends on many factors, including those described above. Generally speaking, the number of amino acid substitutions, insertions or deletions for any given Gas6 polypeptide, fragment or variant will not be more than 40, 30, 20, 19, 18, 17, 16, 15, 14, 13, 12, 11, 10, 9, 8, 7, 6, 5, 4, 3, 2, 1, such as 1-30 or any range or value therein, as specified herein.

Amino acids in a Gas6 peptide of the present invention that are essential for function can be identified by methods known in the art, such as site-directed mutagenesis or alanine-scanning mutagenesis (e.g., Ausubel, supra, Chapters 8, 15; Cunningham and Wells, Science 244:1081-1085 (1989)). The latter procedure introduces single alanine mutations at every residue in the molecule. The resulting mutant molecules are then tested for biological activity, such as, but not limited to at least one Gas6 biological activity. Such biological activity can consist of such as receptor binding or phosphorylation, where the receptors are Axl, Mer or Sky.

As discussed above, minor variations in the amino acid sequences of Gas6 polypeptide molecules are contemplated as being encompassed by the present invention, providing that the variations in the amino acid sequence maintain at least 75%, more preferably at least 80%, 90%, 95%, and most preferably 99% of the amino acids sequences of mammalian Gas6 polypeptide. In particular, conservative amino acid replacements are contemplated. Conservative replacements are those that take place within a family of amino acids that are related in their side chains. Genetically encoded amino acids are generally divided into families: (1) acidic=aspartate, glutamate; (2)basic=lysine, arginine, histidine; (3) non-polar=alanine, valine, leucine, isoleucine, proline, phenylalanine, methionine, tryptophan; and (4) uncharged polar=glycine, asparagine, glutamine, cysteine, serine, threonine, tyrosine. More preferred families are: aliphatic-hydrox=serine, threonine; amide-containing=asparagine, glutamine; aliphatic=alanine, valine, leucine, isoleucine; aromatic=phenylalanine, tryptophan, tyrosine. For example, it is reasonable to expect that an isolated replacement of a leucine with an isoleucine or valine, an aspartate with a glutamate, a threonine with aserine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Assays are described in detail herein.

Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. (Bowie et al. Science 253:164 (1991)). Thus, the foregoing examples demonstrate that those of skill in the art can recognize sequence motifs and structural conformations that may be used to define structural and functional domains in accordance with the invention.

Preferred amino acid substitutions are those which: (1) reduce susceptibility to proteolysis, (2)reduce susceptibility to oxidation, (3) alter binding affinity for forming protein complexes, (4) alter binding affinities, and (4) confer or modify other physicochemical or functional properties of such analogs. Analogs can include various muteins of asequence other than the naturally occurring peptide sequence. For example, single or multiple amino acid substitutions (preferably conservative amino acid substitutions) may be made in the naturally occurring sequence (preferably in the portion of the polypeptide outside the domain(s) forming intermolecular contacts.

A conservative amino acid substitution should not substantially change the structural characteristics of the parent sequence (e.g., a replacement amino acid should not tend to break a helix that occurs in the parent sequence, or disrupt other types of secondary structures that characterize the parent sequence).

(Examples of art-recognized polypeptide secondary and S tertiary structures are described in Creighton, Ed., Proteins, Structures and Molecular Principles W. H. Freeman and Company, New York 1984; C. Branden and J. Tooze, eds., Introduction to Protein Structure Garland Publishing, New York, N.Y. 1991; Thornton et at. Nature 354:105 1991, which are each incorporated herein by reference.)

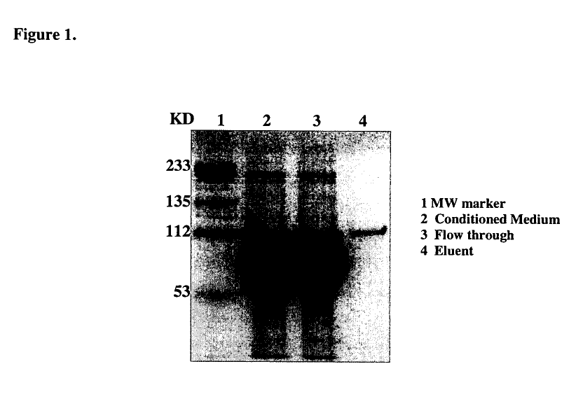

B. Expression and Purification of Epitope-Tagged Gas6 in Mammalian Cells A plasmid vector pcDNA3. 1/Gas6_flag was transfected into human embryonic kidney (HEK) 293 cells by SuperFect Reagent (QIAGEN, Valencia, Calif.) following the manufacture's instruction. The cells were grown in DMEM supplemented with 10% FBS, 2 mM L-glutamine, 100 μ/ml of penicillin and 100 μg/ml of streptomycin. The cell clones were selected in the presence of 600 μg/ml hygromycin B. Drug resistant colonies were picked and screened for protein expression by immunoblotting using both anti-Gas6 (Santa Cruz Biotechnology, Santa Cruz, Calif.) and anti-Flag antibodies (Sigma, St. Louis, Mo.). Positive clones were maintained in the growth medium containing 500 μg/ml of hygromycin B and 10 μg/ml of Vitamin K1. Conditioned media from the cell culture were stored in the presence of 5 mM benzamidine and loaded onto an anti-Flag M2 affinity gel (Sigma, St. Louis, Mo.). Gas6_Flag polypeptide was eluted from the column using the Flag peptide according to the manufacture's instruction. Following dialysis, the protein was concentrated and subjected to analysis. FIG. 1 shows the result from an SDS-PAGE analysis of the purified sample. A single band of ˜75 KD was detected on the gel and was recognized by anti-Flag antibody or anti-Gas6 antibodby following Western blot analysis. The purified Gas6_Flag polypeptide (Seq ID NO.1 and NO. 2) was confirmed by peptide mapping using Voyager System 1092 (Applied Biosystems, Foster City, Calif.) while the N-terminal sequence was determined using Precise Sequencer 1092 (Applied Biosystems, Foster City, Calif.).

As those of skill will appreciate, the present invention includes all other methods of expressing a mamallian recombinant Gas6 protein or polypeptide.

C. Characterization of Epitope-Tagged Gas6 Recombinant Protein or Polypeptides

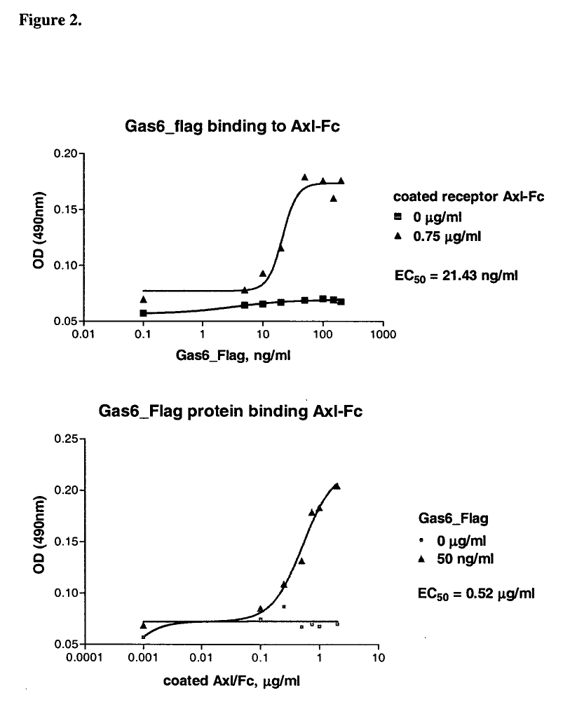

In the present invention, an EIA was used to determine the ability of epitope-tagged Gas6 polypeptide to bind to known Gas6 receptors. Briefly, a 96 well plate was coated with 0.1-2 μg/mL Axl-Fc (R&D Systems, Minneapolis, MN) overnight at 4° C. Then, the plate was washed and blocked with 1% BSA for one hour at RT, incubated with human Gas6_Flag or rat Gas6_Myc-His at 5-200 ng/ml for two hours, and finally followed by treatment with HRP-labeled detecting antibody (anti-Flag M2 antibody or anti-Myc antibody) for one hour. Following incubation with the substrate solution, OD₄₉₀ was read using an automated plate spectrophotometer. FIG. 2 shows the result from human Gas6_Flag. Gas6_Flag dose-dependent bound to Axl-Fc with EC₅₀ around 20 ng/ml, which is comparable to that of non-chimeric Gas6 disclosed by R&D Systems. At 2 μg/ml of Axl-Fc, a native form of recombinant human Gas6 was reported to bind at a linear range of 5-20 ng/ml (R&D Systems, Minneapolis, Minn.). The present invention further includes using recombinant receptors, soluble receptor-Fc fusion or cells expressing any of the recptors to determine Gas6 reactivity to any of the three receptors of this invention.

Epitope-tagged Gas6 protein or polypeptide reactivity to the receptor may also be assessed using an RIA. A 96 well plate may be coated with any of the three receptors, soluble receptor-Fc fusion or cells expressing any of the receptors and probed with ¹²⁵I-Gas6_tag. Bound signals may be quantitated using a gamma counter. Binding characteristics for chimeric Gas6 can be measured by a receptor capture EIA and BIAcore technology.

Epitope-tagged Gas6 polypeptide was tested for its ability to stimulate known Gas6 receptor phosphorylation. Briefly, A172 cells or Du145 cells were treated with epitope-tagged Gas6 for 15 min, lysed and transferred to a 96 well plate coated with anti-Axl antibody (R&D Systems, Minneapolis, Minn.). The plate was probed with a detecting anti-phosphotyrosine antibody 4G10 (preferably HRP-conjugated). Gas6_tag dose-dependent stimulated Axl phosphorylation as expected for native Gas6 protein. The present invention provides other methods of measuring Gas6 biological activities including, but are not limited to, Gas6-induced receptor down regulation and Gas6-dependent cell proliferation.

D. Using Epitope-Tagged Gas6 to Identify Antagonists for Gas6 or Its Receptors

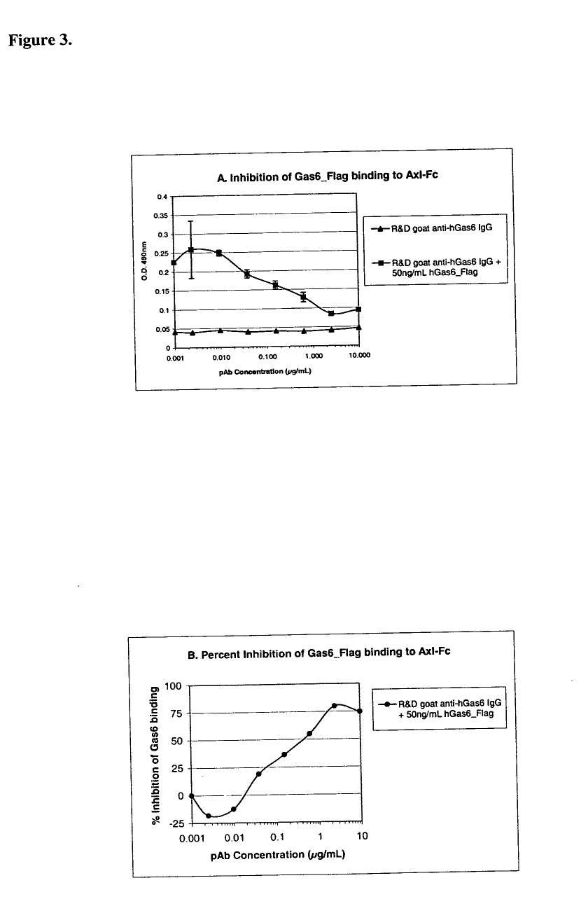

In the present invention, anti-Gas6 antibody (R&D Systems, Minneapolis, Minn.) was used to establish an assay to identify antagonists for Gas6 or its receptors by an EIA. Briefly, a 96 well plate was coated with 0.5 μg/mL Axl-Fc (R&D Systems, Minneapolis, Minn.) overnight at 4° C. The plate was washed and blocked with 1% BSA for one hour at RT and then incubated with Gas6_Flag at 20-50 ng/ml in the presence of 0.01-30 μg/ml anti-Gas6 antibody for two hours. The plate was then treatment with HRP-labeled anti-Flag antibody (Sigma, St. Louis, Mo.) for one hour followed by incubation with a substrate solution. OD₄₉₀ was read using an automated plate spectrophotometer. As seen in FIG. 3, anti-Gas6 antibody dose-dependent inhibited binding of Gas6_Flag to Axl-Fc with an IC₅₀ of ˜0.3 μg/ml. The same antibody was reported to inhibit binding of 10 ng/ml of a native form of human Gas6 to 2 μg/ml immobilized Axl-Fc at an IC50 of ˜1 μg/ml (R&D Systems, Minneapolis, Minn.).

While Gas6 binding to its receptor (Axl, Mer or Sky) is routinely used for screening antagonists, the invention is not so limited. This invention provides other assays including, but not limited to, Gas6-dependent receptor phosphorylation, receptor internalization, cell proliferation, prevention of cell apoptosis, and induction of signaling molecules or cell markers. These may be adapted using an epitope-tagged Gas6 protein or polypeptide as described herein.

### EXAMPLE OF INVENTION

Having generally described the invention, the same will be more readily understood by reference to the following example, which is provided by way of illustration and is not intended as limiting.

It will be clear that the invention can be practiced otherwise than as particularly described in the foregoing description and example.

Numerous modifications and variations of the present invention are possible in light of the above teachings and, therefore, are within the scope of the appended claims.

### EXAMPLE

Generating and Using an Epitope-Tagged Recombinant Gas6 Polypeptide

Epitope-tagged recombinant Gas6 polypeptides that retain their biological activity can be used in compositions where the presence of Gas6 is needed and/or in assays for identifying antagonists for Gas6 and its receptors. To that end and as described herein, a novel method of generating and purifying epitope-tagged mammalian recombinant Gas6 protein or polypeptide is employed.

First, human Gas6 cDNA is obtained by PCR from reverse transcribed human CHRF cDNA, from DNA synthesis, or from any cDNA or genomic library prepared with tissue believed to possess Gas6 mRNA. The cDNA is then inserted into a plasmid, such as pcDNA3.1, which has a hygromycin resistant gene. The reulting plasmid vector is pcDNA3.1/Gas6_Flag

Second, the plasmid vector must be transfected into human embryonic kidney 293 cells. The cells can be grown and then the cell clones can be selected in the presence of hygromycin B. The Gas6_Flag polypeptide can be eluted from a column using the Flag peptide. Once purified, the polypeptide can be confirmed using peptide mapping.

Third, and following purification, the epitope-tagged Gas6 polypeptide will retain its biological activity. The characterization of the tagged polypeptide can be determined by an EIA or RIA, by its ability to stimulate Gas6 receptor phosphorylation, by Gas6-induced receptor down regulation, and by Gas6-dependent cell proliferation.

Fourth, and finally, the epitope-tagged Gas6 polypeptide can be used for at least two, but not limited to two, purposes. The first purpose includes using the tagged polypeptide in compositions where the presence of Gas6 is needed. The second purpose includes using the the tagged polypeptide so as to create an anti-Gas6 antibody that can be used to establish an assay to identify antagonists for Gas6 or for its receptors.

### REFERENCES

1. Schneider, C., R. M. King, and L. Philipson, Genes specifically expressed at growth arrest of mammalian cells. Cell, 1988. 54: p. 787-793.

2. Manfioletti, G., et al., The protein encoded by a growth arrest-specific gene (gas6) is a new member of the vitamin K-dependent proteins related to protein S, a negative coregulator in the blood coagulation cascade. Mol Cell Biol, 1993. 13(8): p. 4976-85.

3. Joseph, D. R., Sequence and functional relationships between androgen-binding protein/sex hormone-binding globulin and its homologs protein S, Gas6, laminin, and agrin. Steroids, 1997. 62(8-9): p. 578-88.

4. Stitt, T. N., et al., The anticoagulation factor protein S and its relative, Gas6, are ligands for the Tyro 3/Axl family of receptor tyrosine kinases. Cell, 1995. 80(4): p. 661-70.

5. Varnum, B. C., et al., Axl receptor tyrosine kinase stimulated by the vitamin K-dependent protein encoded by growth-arrest-specific gene 6. Nature, 1995. 373(6515): p. 623-6.

6. Nagata, K., et al., Identification of the product of growth arrest-specific gene 6 as a common ligand for Axl, Sky, and Mer receptor tyrosine kinases. J Biol Chem, 1996. 271(47): p. 30022-7.

7. Godowski, P. J., et al., Reevaluation of the roles of protein S and Gas6 as ligands for the receptor tyrosine kinase Rse/Tyro3. Cell, 1995. 82(3): p. 355-8. 1 8. Ohashi, K., et al., Stimulation of sky receptor tyrosine kinase by the product of growth arrest-specific gene 6. J Biol Chem, 1995. 270(39): p. 22681-4.

9. Nakano, T., et al., Requirement of gamma-carboxyglutamic acid residues for the biological activity of Gas6: contribution of endogenous Gas6 to the proliferation of vascular smooth muscle cells. Biochem J, 1997. 323(Pt 2): p. 387-92.

10. Tanabe, K., et al., Roles of gamma-carboxylation and a sex hormone-binding globulin-like domain in receptor-binding and in biological activities of Gas6. FEBS Lett, 1997. 408(3): p. 306-10.

11. Mark, M. R., et al., Characterization of Gas6, a member of the superfamily of G domain-containing proteins, as a ligand for Rse and Axl. J Biol Chem, 1996. 271(16): p. 9785-9.

12. Goruppi, S., et al., The product of a gas6 splice variant allows the release of the domain responsible for Axl tyrosine kinase receptor activation. FEBS Lett, 1997. 415(1): p. 59-63.

13. Nakano, T., et al., Cell adhesion to phosphatidylserine mediated by a product of growth arrest-specific gene 6. J Biol Chem, 1997. 272(47): p. 29411-4.

14. Dormady, S. P., X. M. Zhang, and R. S. Basch, Hematopoietic progenitor cells grow on 3T3 fibroblast monolayers that overexpress growth arrest-specific gene-6 (GAS6). Proc Natl Acad Sci U S A, 2000. 97(22): p. 12260-5.

15. Prieto, A. L., et al., Gas6, a ligand for the receptor protein-tyrosine kinase Tyro-3, is widely expressed in the central nervous system. Brain Res, 1999. 816(2): p. 646-61.

16. Ishimoto, Y. and T. Nakano, Release of a product of growth arrest-specific gene 6 from rat platelets. FEBS Lett, 2000. 466(1): p. 197-9.

17. Angelillo-Scherrer, A., et al., Deficiency or inhibition of Gas6 causes platelet dysfunction and protects mice against thrombosis. Nat Med, 2001. 7(2): p. 215-21.

18. O'Donnell, K., et al., Expression of receptor tyrosine kinase Axl and its ligand Gas6 in rheumatoid arthritis: evidence for a novel endothelial cell survival pathway. Am J Pathol, 1999.154(4): p. 1171-80.

19. Melaragno, M. G., et al., Increased expression of Axl tyrosine kinase after vascular injury and regulation by G protein-coupled receptor agonists in rats. Circ Res, 1998. 83(7): p. 697-704.

20. Carmeliet, P. and A. Luttun, Genetic studies on the role of proteinases and growth factors in atherosclerosis and aneurysm formation. Annals of the New York Acad. of Sci., 2001. 947: p. 124-133.

21. Neubauer, A., et al., Expression of axl, a transforming receptor tyrosine kinase, in normal and malignant hematopoiesis. Blood, 1994. 84(6): p. 1931-41.

22. Neubauer, A., et al., Recent progress on the role of Axl, a receptor tyrosine kinase, in malignant transformation of myeloid leukemias. Leuk Lymphoma, 1997. 25(1-2): p. 91-6.

23. Dirks, W., et al., Expression of the growth arrest-specific gene 6 (GAS6) in leukemia and lymphoma cell lines. Leuk Res, 1999. 23(7): p. 643-51.

24. Graham, D. K., et al., Cloning and mRNA expression analysis of a novel human protooncogene, c-mer. Cell Growth Differ, 1994. 5(6): p. 647-57. 25. Graham, D.K., et al., Cloning and developmental expression analysis of the murine c-mer tyrosine kinase. Oncogene, 1995. 10(12): p. 2349-59.

26. Mark, M.R., et al., rse, a novel receptor-type tyrosine kinase with homology to Axl/Ufo, is expressed at high levels in the brain. J Biol Chem, 1994. 269(14): p. 10720-8.

27. Lu, Q. and G. Lemke, Homeostatic regulation of the immune system by receptor tyrosine kinases of the Tyro 3family. Science, 2001. 293(5528): p. 306-11.

28. Li, R., et al., Identification of Gas6 as a growth factor for human Schwann cells. J Neurosci, 1996. 16(6): p. 2012-9.

29. Goruppi, S., E. Ruaro, and C. Schneider, Gas6, the ligand of Axl tyrosine kinase receptor, has mitogenic and survival activities for serum starved NIH3T3 fibroblasts. Oncogene, 1996. 12(3): p. 471-80.

30. Goruppi, S., et al., Gas6 induces growth, beta-catenin stabilization, and T-cell factor transcriptional activation in contact-inhibited C57 mammary cells. Mol Cell Biol, 2001. 21(3): p. 902-15.

31. Yanagita, M., et al., Gas6 regulates mesangial cell proliferation through Axl in experimental glomerulonephritis. Am J Pathol, 2001. 158(4): p. 1423-32.

32. Yin, J. L., et al., Expression of growth arrest-specific gene 6 and its receptors in a rat model of chronic renal transplant rejection. Transplantation, 2002. 73(4): p. 657-60.

33. Allen, M. P., et al., Novel mechanism for gonadotropin-releasing hormone neuronal migration involving Gas6/Ark signaling to p38 mitogen-activated protein kinase. Mol Cell Biol, 2002. 22(2): p.599-613.

34. Goruppi, S., et al., Requirement of phosphatidylinositol 3-kinase-dependent pathway and Src for Gas6-Axl mitogenic and survival activities in NIH 3T3fibroblasts. Mol Cell Biol, 1997. 17(8): p. 4442-53.

35. Goruppi, S., et al., Gas6-mediated survival in NIH3T3 cells activates stress signalling cascade and is independent of Ras. Oncogene, 1999. 18(29): p. 4224-36.

36. Lee, W. P., et al., Axl-gas6 interaction counteracts ElA-mediated cell growth suppression and proapoptotic activity. Mol Cell Biol, 1999. 19(12): p. 8075-82.

37. Lee, W. P., et al., Akt is required for Axl-Gas6 signaling to protect cells from EIA-mediated apoptosis. Oncogene, 2002. 21(3): p. 329-36.

38. Allen, M. P., et al., Growth arrest-specific gene 6 (Gas6)/adhesion related kinase (Ark) signaling promotes gonadotropin-releasing hormone neuronal survival via extracellular signal-regulated kinase (ERK) and Akt. Mol Endocrinol, 1999. 13(2): p. 191-201.

39. Funakoshi, H., et al., Identification of Gas6, a putative ligand for Sky and Axl receptor tyrosine kinases, as a novel neurotrophic factor for hippocampal neurons. J Neurosci Res, 2002. 68(2): p. 150-60.

40. O'Bryan, J. P., et al., axl, a transforming gene isolated from primary human myeloid leukemia cells, encodes a novel receptor tyrosine kinase. Mol Cell Biol, 1991. 11(10): 10 p. 5016-31.

41. Craven, R. J., et al., Receptor tyrosine kinases expressed in metastatic colon cancer. Int J Cancer, 1995. 60(6): p. 791-7.

42. Wimmel, A., et al., Axl receptor tyrosine kinase expression in human lung cancer cell lines correlates with cellular adhesion. Eur J Cancer, 2001. 37(17): p. 2264-74.

43. Crosier, P. S., et al., Identification of a novel receptor tyrosine kinase expressed in acute myeloid leukemic blasts. Leuk Lymphoma, 1995. 18(5-6): p. 443-9.

44. Taylor, I. C., et al., Mouse mammary tumors express elevated levels of RNA encoding the murine homology of SKY, a putative receptor tyrosine kinase. J Biol Chem, 1995. 270(12): p. 6872-80.

45. Lai, C., M. Gore, and G. Lemke, Structure, expression, and activity of tyro 3, a neural adhesion-related receptor tyrosine kinase. Oncogene, 1994. 9: p. 2567-2578.

46. Ling, L. and H. J. Kung, Mitogenic signals and transforming potential of Nyk, a newly identified neural cell adhesion molecule-related receptor tyrosine kinase. Mol Cell Biol, 1995. 15(12): p. 6582-92.

47. Zhan, F., et al., Comparison of multiple myeloma and chronic lymphocytic leukemia identifies novel patterns of gene expression. Blood, 2001. 98: p. 368a-369a.

48. Lu, Q., et al., Tyro-3 family receptors are essential regulators of mammalian spermatogenesis. Nature, 1999. 398(6729): p. 723-8.

49. Nandrot, E., et al., Homozygous deletion in the coding sequence of the c-mer gene in RCS rats unravels general mechanisms of physiological cell adhesion and apoptosis. Neurobiol Dis, 2000. 7(6 Pt B): p. 586-99.

50. Hall, M.O., et al., Outer segment phagocytosis by cultured retinal pigment epithelial cells requires Gas6. Exp Eye Res, 2001. 73(4): p. 509-20.

51. Scott, R. S., et al., Phagocytosis and clearance of apoptotic cells is mediated by MER. Nature, 2001. 411(6834): p. 207-11.

52. Avanzi, G. C., et al., GAS6, the ligand of Axl and Rse receptors, is expressed in hematopoietic tissue but lacks mitogenic activity. Exp Hematol, 1997. 25(12): p. 1219-26.

53. Compernolle, V., et al., Abnormal erythropoiesis in mice deficient in Gas6. Blood, 2001.98: p. 65a-66a.

54. Nakano, T., et al., Vascular smooth muscle cell-derived, Gla-containing growth-potentiating factor for Ca(2+)-mobilizing growth factors. J Biol Chem, 1995. 270(11): p. 5702-5.

55. Fridell, Y. W., et al., GAS6 induces Axl-mediated chemotaxis of vascular smooth muscle cells. J Biol Chem, 1998. 273(12): p. 7123-6.

56. Nakano, T., et al., Prevention of growth arrest-induced cell death of vascular smooth muscle cells by a product of growth arrest-specific gene, gas6. FEBS Lett, 1996. 387(1): p. 78-80.

57. McCloskey, P., et al., GAS6 mediates adhesion of cells expressing the receptor tyrosine kinase Axl. J Biol Chem, 1997. 272(37): p. 23285-91.

58. Avanzi, G. C., et al., GAS6 inhibits granulocyte adhesion to endothelial cells. Blood, 1998. 91(7): p. 2334-40.

59. Augustine, K. A., et al., Noninsulin-dependent diabetes mellitus occurs in mice ectopically expressing the human Axl tyrosine kinase receptor. J Cell Physiol, 1999. 181(3): p. 433-47.

60. Katagiri, M., et al., Mechanism of stimulation of osteoclastic bone resorption through Gas6/Tyro 3, a receptor tyrosine kinase signaling, in mouse osteoclasts. J Biol Chem, 2001. 276(10): p. 7376-82.

What is claimed is:

1. An isolated Gas6 polypeptide with an epitope tag fused at the C-terminus, said epitope tag comprising a Flag sequence as defined in SEQ ID NO. 2.

2. An isolated Gas6 polypeptide of claim 1 comprising a polypeptide of SEQ. ID NO. 1.

3. An isolated Gas6 polypeptide with an epitope tage fused at the C-terminus, said epitope tag comprising a polyhistidine sequence.

4. An isolated Gas6 polypeptide of claim 3 comprising a polypeptide of SEQ. ID NO. 1.

5. A variant Gas6 polypeptide with an epitope tag fused at the C-terminus, said epitope tag comprising a Flag sequence as defined in SEQ ID NO. 2 and said variant Gas6 polypeptide comprising a polypeptide having at least 75% identity to native human Gas6 protein.

6. A variant Gas6 polypeptide of claim 5 having at least 90% identity to native human Gas 6 protein.

7. A variant Gas6 polypeptide with an epitope tag fused at the C-terminus, said epitope tag comprising a polyhistidine sequence and said variant Gas6 polypeptide comprising a polypeptide having at least 75% identity to native human Gas6 protein.

8. A variant Gas6 polypeptide of claim 7 having at least 90% identity to native human Gas 6 protein.

9. Variant Gas6 of either claim 5 or 7 that maintains the biological activity of a native Gas6 protein or polypeptide, wherein said biological activity is receptor binding or phosphorylation, wherein said receptors are Axl, Mer, or Sky.

10. Variant Gas6 of either claim 5 or 7 that maintains its biological activity of a native Gas6 protein or polypeptide, wherein said biological activity is a cellular or molecular response to Gas6.

11. A recombinant DNA molecule encoding the amino acid sequence of the polypeptide of claim 1.

12. A recombinant DNA molecule encoding the amino acid sequence of the polypeptide of claim 1.

13. A vector comprising the nucleic acid of any one of claim 11 or 12.

14. A host cell comprising the vector of claim 13, wherein said host cell is of mammalian origin.

15. A composition comprising the polypeptide of claim 1 and a pharmaceutically acceptable carrier.

16. A composition comprising the polypeptide of claim 3 and a pharmaceutically acceptable carrier.

17. Use of the composition of claim 15 or 16 for the manufacture of a medicine.

18. Use of the composition of claim 15 or 16 for the identification of inhibitors for Gas6 and its receptors, wherein said receptors are Axl, Mer, or Sky.


SEQUENCE LISTING (3 sequences)

SEQ ID NO: 1 - protein, 686 aa, Homo sapiens
MAPSLSPGPAALRRAPQLLLLLLAAECALAALLPAREATQFLRPRQRRAFQVFEEAKQGHLERECVEELCSREEAREVFENDPETDYFYPRYLDCINKYGSPYTKNSGFATCVQNLPDQCTPNPCDRKGTQACQDLMGNFFCLCKAGWGGRLCDKDVNECSQENGGCLQICHNKPGSFHCSCHSGFELSSDGRTCQDIDECADSEACGEARCKNLPGSYSCLCDEGFAYSSQEKACRDVDECLQGRCEQVCVNSPGSYTCHCDGRGGLKLSQDMDTCEDILPCVPFSVAKSVKSLYLGRMFSGTPVIRLRFKRLQPTRLVAEFDFRTFDPEGILLFAGGHQDSTWIVLALRAGRLELQLRYNGVGRVTSSGPVINHGMWQTISVEELARNLVIKVNRDAVMKIAVAGDLFQPERGLYHLNLTVGGIPFHEKDLVQPINPRLDGCMRSWNWLNGEDTTIQETVKVNTRMQCFSVTERGSFYPGSGFAFYSLDYMRTPLDVGTESTWEVEVVAHIRPAADTGVLFALWAPDLRAVPLSVALVDYHSTKKLKKQLVVLAVEHTALALMEIKVCDGQEHVVTVSLRDGEATLEVDGTRGQSEVSAAQLQERLAVLERHLRSPVLTFAGGLPDVPVTSAPVTAFYRGCMTLEVNRRLLDLDEAAYKHSDITAHSCPPVEPAAADYKDDDDK

SEQ ID NO: 2 - protein, 8 aa, Homo sapiens
DYKDDDDK

SEQ ID NO: 3 - protein, 678 aa, Homo sapiens
MAPSLSPGPAALRRAPQLLLLLLAAECALAALLPAREATQFLRPRQRRAFQVFEEAKQGHLERECVEELCSREEAREVFENDPETDYFYPRYLDCINKYGSPYTKNSGFATCVQNLPDQCTPNPCDRKGTQACQDLMGNFFCLCKAGWGGRLCDKDVNECSQENGGCLQICHNKPGSFHCSCHSGFELSSDGRTCQDIDECADSEACGEARCKNLPGSYSCLCDEGFAYSSQEKACRDVDECLQGRCEQVCVNSPGSYTCHCDGRGGLKLSQDMDTCEDILPCVPFSVAKSVKSLYLGRMFSGTPVIRLRFKRLQPTRLVAEFDFRTFDPEGILLFAGGHQDSTWIVLALRAGRLELQLRYNGVGRVTSSGPVINHGMWQTISVEELARNLVIKVNRDAVMKIAVAGDLFQPERGLYHLNLTVGGIPFHEKDLVQPINPRLDGCMRSWNWLNGEDTTIQETVKVNTRMQCFSVTERGSFYPGSGFAFYSLDYMRTPLDVGTESTWEVEVVAHIRPAADTGVLFALWAPDLRAVPLSVALVDYHSTKKLKKQLVVLAVEHTALALMEIKVCDGQEHVVTVSLRDGEATLEVDGTRGQSEVSAAQLQERLAVLERHLRSPVLTFAGGLPDVPVTSAPVTAFYRGCMTLEVNRRLLDLDEAAYKHSDITAHSCPPVEPAAA